(19) United States
(12) Patent Application Publication
(10) Pub. No.: US 2017/0189875 A1
(43) Pub. Date: Jul. 6, 2017

(54) LOW PRESSURE DROP PACKING MATERIAL STRUCTURES

(71) Applicant: BASF CORPORATION, Florham Park, NJ (US)

(72) Inventors: Dieter G. VON DEAK, Hazlet, NJ (US); Stefan LIPP, Karlsruhe (DE); Christian-Andreas WINKLER, Mannheim (DE); Wolfgang GERLINGER, Limburgerhof (DE)

(73) Assignee: BASF Corporation, Florham Park, NJ (US)

(21) Appl. No.: 15/321,305

(22) PCT Filed: Jun. 24, 2015
(86) PCT No.: PCT/US2015/037476

§ 371 Date: Dec. 22, 2016

Related U.S. Application Data
(60) Provisional application No. 62/017,611, filed on Jun. 26, 2014.

(51) Int. Cl. B01J 8/02; B01J 21/04; B01J 35/02; C07C 1/24; C07C 67/00; C07D 301/03; C07C 45/00; B01J 19/30; C07B 35/04

(52) U.S. Cl. CPC B01J 8/0292; B01J 8/025; B01J 19/30; B01J 21/04; B01J 35/026; C07B 35/04; C07C 67/00; C07D 301/03; C07C 45/00; C07C 1/24; B01J 2208/00805; B01J 2219/30223; B01J 2219/30261; B01J 2219/30242; B01J 2219/30296; B01J 2219/30416; B01J 2219/30475

(57) ABSTRACT

A packed bed includes a vessel including a shell, an inlet, and an outlet, wherein the space inside the shell between the inlet and outlet forms an internal volume; a plurality of packing material structures filling at least a portion of the internal volume thereby forming a packed volume, wherein the packed volume has a void fraction, and the packing material structures provide an aggregate surface area; and the vessel has a pressure drop between the vessel inlet and vessel outlet, wherein the pressure drop is less than 1.0 times that of a packed bed of the non-twisted shapes with the same cross-section.

### CROSS-REFERENCE TO RELATED APPLICATIONS

This application claims priority to U.S. Provisional Application No. 62/017,611, filed Jun. 26, 2014, the contents of which are incorporated by reference in its entirety into the present disclosure.

### TECHNICAL FIELD

Principles and embodiments of the present invention relate generally to shaped packing structures that provide increased surface area and lower pressure drops across a packed space at least partially due to the shapes' resistance to nesting of the packing structure bodies.

### BACKGROUND

Various shapes and types of packing materials have been used for chemical processing. For example, spheres, Raschig rings, Pall rings, Berl saddles, Intalox saddles, and various other random packing have been employed in columns and reactors to provide increased contact surface area and distribution of liquids and gasses.

Various polylobal particles, some having a helical shape, have also been disclosed, for example in U.S. Pat. No. 4,673,664 to Bambrick issued Jun. 16, 1987.

In certain processes there is only the flow of a single fluid phase through a column of stationary solid particles, for example in a fixed-bed catalytic reactor and sorption operations (e.g., adsorption, ion exchange, and ion exclusion.). In other instances, there is a two-phase flow that includes a gas and a liquid, for example in separations (e.g., distillation, absorption, and stripping). The liquid may fill some of the void space in the packing, while the gas travels in a counter current through the remaining void space.

For example, in a packed column used for gas-liquid contact, the liquid flows downward over the surface of the packing and the gas flows upward in the void space of the packing material. A low pressure drop and a very large surface area for mass transfer are important for the performance of such packed towers. The packing material provides the increased surface area for mass transfer interaction and void space for the flow of the process streams, which can result in a pressure drop.

Pressure drop through a packed bed can be caused by both inertia transfer and friction forces between the moving fluids and packing material.

While many different shapes of random packing materials have been investigated, there is still a need for packing material structures that provide greater surface area with lower pressure drops to provide overall increases in packed bed efficiencies and performance.

### SUMMARY

Principles and embodiments of the present invention provide three-dimensional structures that resist the stacking, nesting, and intermeshing that has occurred with previous shapes and structures.

Principles and embodiments of the present invention relate to providing shapes and relationships between the packing material configurations and dimensions in a manner that reduces or eliminates the tendency of the packing material structures to fill the open channels or groves of adjacent structures by stacking or nesting, as well as maintain a greater distance of closest approach to produce a lower pressure drop.

Principles and embodiments of the present invention relate to packing material structures having a twist, a curve, one or more channels, or a combination thereof that reduce the likelihood of adjacent packing material structures assuming orientations that make stacking and nesting favorable, and hampers the nesting and intermeshing of two or more packing material structures, which can result in filling of the structure's open spaces.

Principles and embodiments of the present invention also relates to maintaining the aggregate surface area of a plurality of packing material structures, while reducing the pressure drop across a random packed bed, reactor, tank, drum, column, tower, pipe, duct, or tube.

Principles and embodiments of the present invention also relates to maintaining the pressure drop across a random packed bed, reactor, tank, column, tower, pipe, duct, or tube, while increasing the aggregate amount of active surface area of the plurality of packing material structures.

Principles and embodiments of the present invention relate to a packed bed comprising a vessel comprising a shell, an inlet, and an outlet, wherein the space inside the shell between the inlet and outlet forms an internal volume, a plurality of packing material structures filling at least a portion of the internal volume thereby forming a packed volume, wherein the packed volume has a void fraction, and the packing material structures provide an aggregate surface area, and the vessel has a pressure drop between the vessel inlet and vessel outlet, wherein the pressure drop is less than 1.0 times the pressure drop of a packed bed of the non-twisted shapes with the same or a similar cross-section. In various embodiments, the pressure drop is less than 0.8 times the pressure drop of a packed bed of the non-twisted shapes with the same or a similar cross-section.

In various embodiments, the void fraction is less than about 0.8 and the pressure drop is less than 0.95 times the pressure drop of a packed bed of the non-twisted shapes with the same or a similar cross-section.

In various embodiments of the packed bed, the void fraction is less than about 0.8 and greater than about 0.45, and the pressure drop is less than 0.95 times the pressure drop of a packed bed of the non-twisted shapes with the same or a similar cross-section and greater than about 0.3 times the pressure drop of a packed bed of the non-twisted shapes with the same or a similar cross-section, where the packed bed comprises a plurality of packing material structures with one or more helical channels.

In various embodiments of the packed bed, the void fraction is less than about 0.8 and greater than about 0.55, and the pressure drop is less than 0.8 times the packed bed of the non-twisted shapes with the same or a similar cross-section and greater than about 0.3 times the pressure drop of packed bed of the non-twisted shapes with the same or a similar cross-section, where the packed bed comprises a plurality of packing material structures with one or more helical channels.

In various embodiments of the packed bed, the vessel is a tower, column, tank, drum, tube, pipe, or duct.

Principles and embodiments of the present invention also relates to a packing material structure comprising a body having an external surface having a length (L) and an outer diameter (OD) defining an aspect ratio of L/OD, wherein the aspect ratio is greater than 1 and less than 10, and at least one continuous recess formed in the external surface, wherein the structure is chemically active to absorb or catalyze chemical moieties that contact a surface of the support.

In various embodiments of the packing material structure, the recess rotates around the central axis of the body by an angle of rotation θₗ per unit body length equal to the OD, wherein θₗ is between an about 45° and 180°.

In various embodiments of the packing material structure, the body further comprises at least one hollow bore with a diameter D_{b} though the body, which forms an internal surface, where D_{b}≦OD−4 mm.

In various embodiments of the packing material structure, the recess has a depth of less than Dᵣ, where Dᵣ=(OD−(D_{b}+2))/2.

In various embodiments of the packing material structure, the cross-section of the hollow bore has a non-circular shape.

In various embodiments of the packing material structure, the D_{b} is between about 10% and 50% of the OD.

In various embodiments of the packing material structure, the thickness of a wall T_{w} between the OD and the D_{b} is between about 10% and 40% of the OD.

Principles and embodiments of the present invention also relate to a packed bed comprising a plurality of packing material structures, wherein the plurality of packing material structures have an OD of between about 1.0 mm and about 15.0 mm.

In various embodiments, the packed bed has a pressure drop of less than 0.8 times that of packed bed of the non-twisted shapes with the same or a similar cross-section, and a geometric surface area to reactor volume ratio of greater than 500 m²/m³.

In various embodiments of the packed bed, the packing material structure is composed of alumina, silica, activated carbon, graphitic carbon, single-walled carbon nano-tubes, titanium dioxide, calcium carbonate, barium sulfate, zeolite, cerium oxide, magnesium oxide, or zinc oxide.

Principles and embodiments of the present invention also relate to a packing material structure comprising an extruded body comprising a geometric cross-section with N_{f} edges and Nᵥ vertices, and an axis of extrusion of length L_{b}, wherein the vertices are a distance Rᵥ from the axis and the axis of extrusion traces a path from a first face of the extruded body to a second face of the extruded body, wherein the catalytic support is catalytically active.

In various embodiments of the packing material structure, the extruded body has an aspect ratio (L/OD) of greater than 1 to about 10.

In various embodiments of the packing material structure, the extruded body further comprises a hollow bore through the interior of the extruded body.

In various embodiments of the packing material structure, the N_{f} edges are concave such that the extruded body further comprises N_{c} channels between the Nᵥ vertices, wherein the perimeter of a cross-section of the support is greater than the circumference of a circle having a radius of Rᵥ.

In various embodiments of the packing material structure, the N_{f} edges are convex such that the extruded body further comprises Nₗ lobes between the Nᵥ vertices, wherein the lobes have a maximum distance R_{L} from the axis, and perimeter of a cross-section of the support is greater than the circumference of a circle having a radius of R_{L}.

In various embodiments of the packing material structure, Nᵥ=2x, and x=3 to 8, and wherein the even-numbered vertices are located between the odd-numbered vertices and are a distance Rᵢ from the axis of extrusion and the odd-numbered vertices are a distance Ro from the axis of extrusion, wherein the edges between the vertices form N_{c}/2 channels with a channel depth Dₑ=Rₒ−Rᵢ.

In various embodiments of the packing material structure, the extruded body further comprises a hollow bore through the interior of the extruded body, and the wall thickness Tw between the diameter of the hollow bore R_{b} and Ri is at least 1 mm.

In various embodiments of the packing material structure, the channel depth Dc is from between about 0.1 mm to about 3.0 mm

In various embodiments of the packing material structure, the body is extruded along a curved axis of extrusion, wherein the catalytic support is C-shaped.

In various embodiments of the packing material structure, the N_{f} edges and Nᵥ vertices are twisted around the axis of extrusion so that they have an angle of rotation θₗ per unit body length of OD to form at least three helical shaped channels or grooves wound about the axis of extrusion along the length of the particle.

In various embodiments of the packing material structure, the angle of rotation θₗ per unit body length of OD of the twisted N_{f} edges and Nᵥ vertices is between about 45° and 180°.

Principles and embodiments of the present invention relate to a geometrically shaped solid comprising a solid comprising, a cylindrical body formed by a surface of revolution around a curved axis of revolution, wherein the surface of revolution is at a distance R₁ from the axis of revolution in a plane perpendicular from the axis of revolution, one or more channels circumscribing a helical path around the cylindrical body, wherein the one or more channels have a crest at the surface of revolution of the cylindrical body and a trough within the cylindrical body, and the trough is at a distance R₂ from the axis of revolution, and a length L_{b} extending from a first face of the cylindrical body to a second face of the cylindrical body, wherein the shaped solid is catalytically active.

In various embodiments of the shaped solid, the axis of revolution has a varying radius R₀, which changes over an arc of angle L₀.

In various embodiments of the shaped solid, the axis of revolution has a constant radius R₀, and circumscribes an arc of angle L₀.

In various embodiments of the shaped solid, the curved cylindrical body has the shape of a segment of a torus with one or more channels formed around the body, such that the pitch between the channels along the outside edge of the torus is greater than the pitch along the inside edge of the torus.

### BRIEF DESCRIPTION OF THE DRAWINGS

Further features of the various embodiments of the present invention, their nature and various advantages will become more apparent upon consideration of the following detailed description, taken in conjunction with the accompanying drawings, which are also illustrative of the best mode contemplated by the applicants, and in which like reference characters refer to like parts throughout, where:

FIG. 1 illustrates an exemplary packed column in a vertical orientation;

FIGS. 2A-B illustrate exemplary embodiments of packing material structures;

FIGS. 3A-C illustrate exemplary embodiments of packing material structures having at least one recess;

FIGS. 4A-C illustrate exemplary embodiments of packing material structures with a geometric cross-sectional shape;

FIGS. 5A-B illustrate exemplary embodiments of packing material structures with concave or convex edges;

FIGS. 6A and 6B illustrate another exemplary embodiment of a packing material structure;

FIGS. 7A-B illustrate other exemplary embodiments of a packing material structure;

FIG. 8 illustrates another exemplary embodiment of a packing material structure;

FIG. 9 illustrates an exemplary embodiment of a curved packing material structure;

FIG. 10 illustrates another exemplary embodiment of a curved packing material structure; and

FIG. 11 illustrates another exemplary embodiment of a curved packing material structure.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Principles and embodiments of the present invention relate to packing material structures having particular shapes, sizes, and configurations, which provide increased surface area and/or produce a reduced pressure drop in a randomly packed column, bed, tube, drum, reactor, tower, tank, duct, or pipe.

Principles and embodiments of the present invention relate to packing material structures that may be employed in physical and chemical interactions, including but not limited to physisorptions, chemisorptions, adsorption-desorptions, chromatography, ion exchange, distillations, surface-promoted reactions, and catalytically activated processes.

Embodiments of the packing material structures can provide surfaces for mass transfer processes and catalysis, where larger surface areas may increase throughput for surface limited interactions.

Principles and embodiment of the present invention relate to packing material structures that may be employed in a wide variety of thermal or catalytically activated and/or enhanced processes, including, for example, cracking, reforming, hydrogenation, oxidation, dehydrogenation, dehydration, polymerization, alkylation or dealkylation of aryl compounds, for example including benzene; the isomerization of various materials, including, for example, xylene; hydrodesulfurization; and the conversion of substances, such as coal-derived compounds or methanol or other hydrocarbons into materials, such as olefins, fuels, or lubricants, and the like. The particular configuration of the formed packing material has been developed to enhance the catalytic activity and physical properties, such as pressure drop, surface area, crush strength and abrasion resistance of the particle, as well as the selectivity of the catalyst for the particularly desired product. The shaped packing material structures of the various embodiments may also be employed in applications, including guard bed service and/or as catalyst supports. Furthermore, the embodiments of the present invention may be exposed to and/or interact with gases, liquids, suspended solids, multi-phase components, or a combination thereof.

In embodiments of the present invention, the packing material structures may be comprised of a material that is catalytically active, have catalytically active materials deposited on the surface of the structure, or a combination of both, where catalytically active refers to compositions that promote, enhance, and/or initiate a reaction. When the packing material structure comprises catalytically active material(s), the structure may be considered to be catalytic support.

Catalytically active materials may include noble metals, base metals, metal oxides, alkali metals, platinum group metals, or a combination thereof.

In embodiments of the present invention, the packing material structures may be comprised of a material that has binding sites, have active materials deposited on the surface of the structure that provide binding sites, or a combination of both, where binding sites refer to surface features that promote or enhance the chemical or physical absorption of chemical species to the surface of the packing material structure.

In embodiments of the present invention, the packing material structures may be comprised of a porous material that provides increased surface area for absorption and/or catalytic activity, where the increased surface are may be provided by pores and/or channels having a broad or narrow range of sizes within the packing material structure.

In embodiments of the present invention, the shapes of the packing material structures can limit the extent to which each body can nest with another neighboring body due to surface features and/or configurations that interfere with a protruding portion of the first body entering a channel or other concavity in the neighboring body.

In embodiments of the invention, the pressure drop across a reactor can be lessened for the same packing material and/or catalyst diameter by twisting the packing material structure around a longitudinal axis. In addition, by using the same shape at a smaller diameter to achieve the same pressure drop considerably more catalyst geometric surface area can be added to the same reactor volume. The various embodiments, provide a balance between the amount of packing material, the resulting surface area, and the resulting pressure drop, so that more utilized packing material can provide more surface area, while maintaining the same pressure drop, or the same surface area can be provided with less packing material and a lower pressure drop, or a combination of a reduction in packing material and reduction in pressure drop can be provided.

Pressure drop should be understood to be the difference in pressure between two points of a fluid carrying network. For example, if the fluid carrying network is a cylindrical packed bed of catalyst particles, with the fluid being air at standard temperature and pressure, the pressure drop would be determined by measuring the pressure difference across a packed bed while flowing fluid at various rates in the laminar and/or turbulent flow regimes through the packed bed. In a non-limiting example, the diameter of the tube can be greater than 10 times the outer diameter of the packing particles and the length of the tube can be equal to or greater than 50 times the outer diameter of the packing particles in order to diminish the effect of edges. For various embodiments of the twisted shapes, a pressure drop should be <0.3 psig/ft with air flowing at 3 ft/s at 25° C. entering at 1 atm.

Principles and embodiments of the present invention relate to a packing material structure that provides a void space fraction of less than about 0.8, or alternatively between about 0.8 and about 0.45, or between about 0.55 and 0.8, where the void fraction, also referred to as void space fraction, is a measure of the empty space in the packed portion of a bed, and is the volume of empty space over the total volume of the bed portion, which can have a value of between 0 and 1.

The Reynolds number for a packed bed is given by:

$$Re = \frac{ρ {v}_{s} D}{μ} .$$

where D is the particle diameter of the packing, ρ is the density of the fluid flowing through the bed, vₛ is the superficial velocity of the fluid determined by the volume flow rate divided by the cross-sectional area of the bed, and μ is the dynamic viscosity of the fluid. For turbulent flow, the Reynolds Number is >100.

When the packing has a shape different from spherical, an effective particle diameter is given by:

Dₚ=6Vₚ/Aₚ=6(1−ε)/Aₛ

Aₛ is the interfacial area of packing per unit of packing volume, ft²/ft³ or m²/m³.

The Ergun equation relates that pressure drop to void space, and can be given by:

$${f}_{p} = \frac{150}{{Gr}_{p}} + 1.75$$

where fₚ and Grₚ are defined as:

$${f}_{p} = \frac{Δ p}{L} \frac{{D}_{p}}{ρ {V}_{s}^{2}} ( \frac{{c}^{3}}{1 - ε} ) and {Gr}_{p} = \frac{{D}_{p} {V}_{s} ρ}{( 1 - ε ) μ}$$

where: Grₚ is a form of Reynolds number for fluidized beds, Δp is the pressure drop across the bed, L is the length of the bed, Dₚ is the equivalent spherical diameter of the packing, ρ is the density of fluid, μ is the dynamic viscosity of the fluid, Vₛ is the superficial velocity (i.e. the velocity that the fluid would have through the empty tube at the same volumetric flow rate), and ε is the void fraction of the bed (bed porosity at any time). The void fraction may also be referred to as a void space or void space fraction.

The equation can be rearranged to represent the direct relationship of void fraction or void space to pressure drop.

$$Δ p = \frac{150 {μ ( 1 - ε )}^{2} {V}_{s} L}{{ε}^{3} {D}_{p}^{2}} + \frac{1.75 ( 1 - ε ) ρ {V}_{s}^{2} L}{{ε}^{3} {D}_{p}}$$

As can be seen, the pressure drop Δp is related to the ratio of (1−ε)²/ε³, where the ratio is approximately 1 at ε=0.57, and (1−ε)/ε³. In addition, the pressure drop Δp is related to 1/Dₚ² and 1/Dₚ respectively. The first term to the right of the equal sign is generally dominant for laminar flow regimes, while the second term is generally dominant in the turbulent flow regimes. Furthermore, for high rates of flow, the first term drops out, whereas at low rates of flow the second term drops out.

In embodiments, the pressure drop per unit length of a packed bed is generally inversely proportional to the size of the particle raised to a power of one (1) or greater, so that the pressure drop generally may be reduced by using a larger particle size. The size of the particle has a greater affect at lower flow rates.

Furthermore, the pressure drop per unit length of a packed bed is generally proportional to the void space term, 1/ε³.

Embodiments of the present invention relate to packing material structures that increase both the void fraction ε and the effective particle diameter Dₚ. to produce a lower pressure drop for the same length of packed bed. The effective particle diameter Dₚ may be affected by changes in the aspect ratio L/OD of the packing material structure.

In embodiments, the packing material structure can have a shape factor φₛ that is defined as the ratio of the surface area of a sphere with the material volume equal to the volume of the packing material structure divided by the actual surface area of the packing material structure.

In a non-limiting embodiment, for example, a hexagonal prism within a circumscribed circle with a diameter of 1 cm and a length of 2.5 cm, the perimeter is 6 cm, the surface area of each end is 3 cm², the surface area of the structure is 21 cm², and the volume is 7.5 cm³. A sphere with a volume of 7.5 cm³ has a diameter of 2.4286 cm, and a surface area of 18.5294 cm². The resulting shape factor φₛ is about 0.8823. In comparison, a sphere has a shape factor φₛ of 1, and a cylinder with a diameter of 1 cm and a length of 2.5 cm has volume of 7.854 cm³, an area of 21.991 cm², and a shape factor φₛ of about 0.8689. Lower pressure drops have been correlated with larger shape factors. As can be seen for the non-limiting example, the polygonal shape has a larger shape factor than a related cylindrical shape of similar dimensions resulting from the comparable surface area but reduced volume. Due to the reduced volume, a greater aggregate surface area may be provided by the hexagonal packing structure filling the same packed bed volume.

In embodiments of the present invention, the packing material structures may have a catalytic material deposited on the surface of the packing material structures, where the catalytic material may be noble metals, base metals, metal oxides, ion-exchange compounds and resins, chelating resins, electron-exchange resins, activators, and promotors.

Embodiments of the present invention also relate to a hydrocarbon conversion catalyst comprising a packing material structure having a shape which can provide high surface areas with reduced probability of interlocking, wherein a plurality of packing material structures may be employed in any fixed bed catalytic process including conversion of hydrocarbonaceous feedstocks, for example, isomerization, alkylation, reforming, and hydroprocessing, including hydrocracking, hydrotreating, hydrofining, hydrodemetalation, hydrodesulfurization, and hydrodenitrogenation. The packing material structures may support a catalytically active material that promotes or enhances hydrocracking, hydrotreating, hydrofining, hydrodemetalation, hydrodesulfurization, and hydrodenitrogenation.

For example, the packing material structures may be applied to the production of vinyl acetate monomers, conversion of ethylene to ethylene oxide, and the conversion of alcohols to carbonyls.

As used herein, reference to a bed or packed bed shall mean vessels including but not limited to towers, columns, tanks, drums, tubes, pipes, ducts, and other containers that can include packing material to increase surface area and/or provide support to catalytic material(s) for chemical and physical processes, where the packing material may be retained within an internal space of the vessel, for example reactors, tanks, towers, columns, pipes, tubes, ducts, and other containers.

An effective packing surface area can depend on at least the size, shape, and configuration of the packing material structures, and may be less than the total theoretical aggregate area of the plurality of packing material structures due to nesting and/or distribution in a packed bad.

Embodiments of the present invention also relates to a packed bed comprising a hollow structure having an internal volume and a plurality of packing material structures filing at least a portion of the internal volume, wherein the packed bed pressure drop is less than 0.8 times the pressure drop of a packed bed of the non-twisted shapes with the same or a similar cross-section.

A value for the pressure drop may be normalized by establishing a reference value and dividing all other values by the reference value. For example, in various embodiments, the pressure drop may be normalized by dividing the helical channel shaped packing material structures by the value obtained for a spherical packing material with a hydrodynamic particle diameter comparable to the helical shaped packing material structures. In some embodiments, the reference value is determined for a packing material structure having the same diameter, aspect ratio, and depth of channel, (e.g., the same cross-section), but having a straight profile (e.g., 0° twist) instead of a helical shape (e.g., >0° twist).

In some embodiments, a helical shaped packing structure having intermediate values for diameter, aspect ratio, depth of channel, and degree of twist may be chosen as the reference structure for comparison with all other evaluated structures.

In other embodiments, the values for various helical shaped packing structures may be normalized against the average of all evaluated helical shaped packing structure values to obtain an internal reference to generate relative (i.e., comparative) values between each of the different shapes.

In embodiments of the invention the void space of the packed bed may be less than about 0.8 but greater than about 0.45, or alternatively the void space may be between about 0.60 and about 0.75.

Embodiments of the present invention relate to a packing material structure comprising a body with an external surface having a length (L) and an outer diameter (OD) defining an aspect ratio of L/OD, wherein the aspect ratio may be in the range of greater than 1 and less than 10, or alternatively greater than 1 and less than 5, or greater than 1 and less than 2. The various embodiments have a greater surface area and/or greater shape factor than a cylinder having comparable dimensions of length and diameter.

Principles and embodiments of the present invention relate to packing material structures that have a transverse cross-section with an outer perimeter that is longer than the circumference of a circle that would circumscribe the cross-sectional shape.

In various embodiment of the present invention, the packing material structure may be made of alumina, silica, activated carbon, graphitic carbon, single-walled carbon nano-tubes, titanium dioxide, calcium carbonate, barium sulfate, zeolite, cerium oxide, magnesium oxide, or zinc oxide.

In various embodiments, the packing material structure may be extruded through a die and cut to desired lengths. In some embodiments, a curve or C-shape may be imparted to the extruded shape by deflecting the extrudate and/or draping it over or spooling it around a mandrel of diameter D_{M}.

Previously known packing materials used in random packed beds, reactors, columns, towers, tanks, pipes, ducts, and tubes for various types of chemical processing have had a tendency for the external features of the material that provide an increased surface area to intermesh or nest in a manner that significantly reduces or eliminates the void space between individual packing material structures with a resulting increase in pressure drop along with a decrease in active surface area. Such nesting or intermeshing has also been recognized as contributing to channeling, increased pressure drops, and lower flood points in columns and reactors.

Applicants have determined that cylindrical, star, lobed and geometric (e.g., triangular, square, rectangular, pentagonal, hexagonal, etc.) shaped packing materials with straight features (e.g., a prism) will pack together in a reactor, tower, tank, column, pipe, tube, or bed, such that the external surfaces will contact each other in a manner that decreases the aggregate active surface area and void space. Furthermore, such packing or nesting results in a smaller distance of closest approach between two or more packing structures, so the material packs more tightly in a bed and takes up less volume, thereby requiring more packing material to fill the bed volume and adding to the cost.

In a particular instance, for example, the points of one star-shaped packing structure can intermesh with the space between the points of a neighboring star-shaped packing structure in the manner of two intermeshing gears. This nesting of two or more packing structures results in the projections of one structure filling the spaces intended to be created by the gaps in the other structure, and allowing two otherwise active surfaces to contact each other, thereby at least partially defeating the purpose of having packing structures with complex profiles and increased surface areas.

In addition, in particular instances, the flat end face of one packing structure will abut the flat end of an adjacent packing structure, which can result in further reduction of active surface area due to direct contact between the end faces, and possible blocking of access to internal bores and channels.

Various exemplary embodiments of the invention are described in more detail with reference to the figures. It should be understood that these drawings only illustrate some of the embodiments, and do not represent the full scope of the present invention for which reference should be made to the accompanying claims.

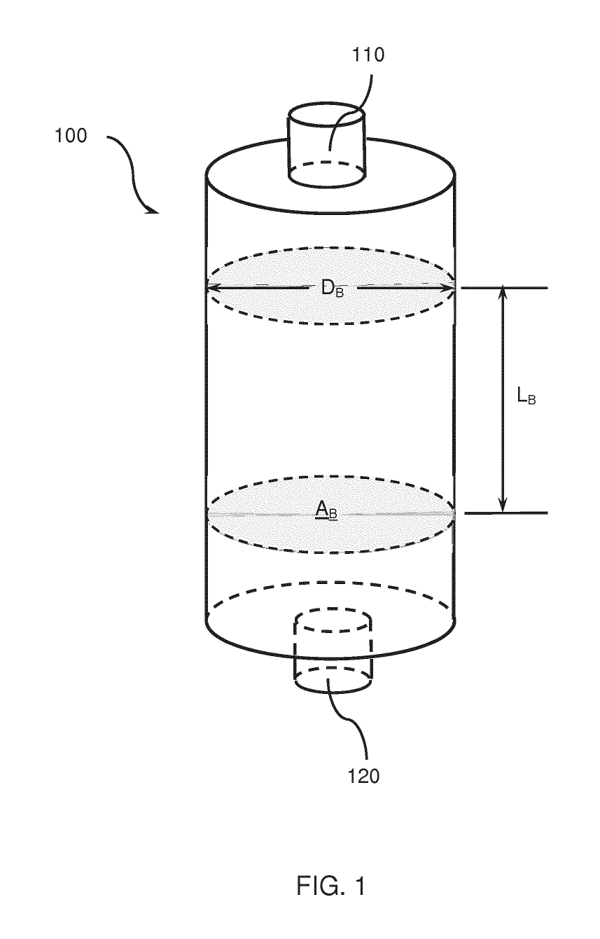

FIG. 1 illustrates an exemplary packed column 100 having a vertical orientation in which at least a portion of the internal volume may be filled with a packing material. The section of the internal volume containing the packing material has a length L_{B}, a diameter D_{B} and an area A_{B} that may be related to the pressure drop between the inlet and outlet of the column 100. The packing material may be retained within a specific portion or section of the internal volume by utilizing various support plates and retainers 140 known in the art of chemical engineering and unit operations. The various vessels may also comprise distributors, separators, and reactor internals known in the art. It should be recognized that while the inlet 110 is shown at the top of the column and the outlet 120 is shown at the bottom of the column, the arrangement may be reversed depending upon the phase of the fluid(s) (e.g., liquid or gas), the density of the fluid (e.g., water, organic; hot, cold) being introduced to the packed bed and the role that gravity may play in the chemical or physical process implementing the packed bed. Similarly, it should be understood that while the illustrated column is shown in a vertical orientation, packed beds may be implemented in pipes, tubes, reactors and ducts with a horizontal orientation. Various retainers and packing methods may be employed to implement a packed bed with different orientations, as is known in the chemical engineering arts.

In various embodiments, the packed bed may have a volume V_{B}, that is loaded with a plurality of packing material structures where the fraction of open space after packing is the void fraction or void space, E, discussed above in reference to the Ergun equation. A packed bed having a particular void space fraction ε can experience a pressure drop due to frictional and inertial losses by the flowing fluid(s).

In various embodiments of the packed bed of the present invention, the void space fraction produced by the packing material structures may be between about 0.45 and about 0.8.

In various embodiments, the plurality of packing material structures loaded into the packed bed volume, V_{B}, will provide an aggregate surface area, A_{P}, where the aggregate surface area may be calculated by multiplying the number of packing material structures by the surface area per packing material structure Aₛ. This can provide a surface area per volume A_{P}/V_{B} (m²/m³) for the packed bed.

The various embodiments may provide a void fraction and a pressure drop in a packed bed, where the pressure drop is less than 0.95 times the pressure drop of a packed bed of the non-twisted shapes with the same or a similar cross-section, and the void fraction is between about 0.50 and 0.75.

Principles and embodiments of the present invention also relate to the relationship between the geometric properties of the packing material structures and the performance characteristics of a packed bed comprising a plurality of such packing material structures, where the geometric properties include but are not limited to the OD, the aspect ratio, the number of recesses, the depth of the recesses, the angle of twist of the recesses, and the presence of a bore in the structure. The packed bed performance characteristics may include but not be limited to the surface area per packed bed volume, the pressure drop of the packed bed, and the void space fraction of the packed bed.

In various embodiments, the pressure drop and void fraction may be related to the packing structure OD, length, and angle of twist or rotation.

In embodiments of the invention the void space of the packed bed may be between about 0.60 and about 0.75, and the pressure drop may be between about 0.3 and about 0.95 times that of a packed bed of the non-twisted shapes with the same or a similar cross-section, for packing material structure having an aspect ratio of greater than 1.0 to about 2.0, 5 N_{f} edges and 5 Nᵥ vertices, and a channel depth of about 0.9 mm.

In embodiments of the invention the void space of the packed bed may be between about 0.65 and about 0.75, and the pressure drop may be between about 0.3 and about 0.8 times that of a packed bed of the non-twisted shapes with the same or a similar cross-section, for packing material structure having an aspect ratio of greater than 1.0 to about 2.0, 5 N_{f} edges and 5 Nᵥ vertices, a twist with a 180° angle of rotation, and a channel depth of about 0.9 mm.

Embodiments of a packed bed having the described pressure drop and void fractions may be achieved utilizing aggregates of the various packing material structures described herein.

In the various embodiments, the twist may be left-handed or right-handed. In some embodiments, a packed bed may comprise both left and right hand twisted packing material structures.

In contrast to a screw which may typically have symmetrical dimensions for the crest, trough, and pitch, such that the crest can fit into the trough of an adjacent screw, the embodiments of the present invention may have a feature equivalent to a screw crest that is larger than the trough (e.g., a channel), so that the crest cannot physically fit within the trough (e.g., a channel).

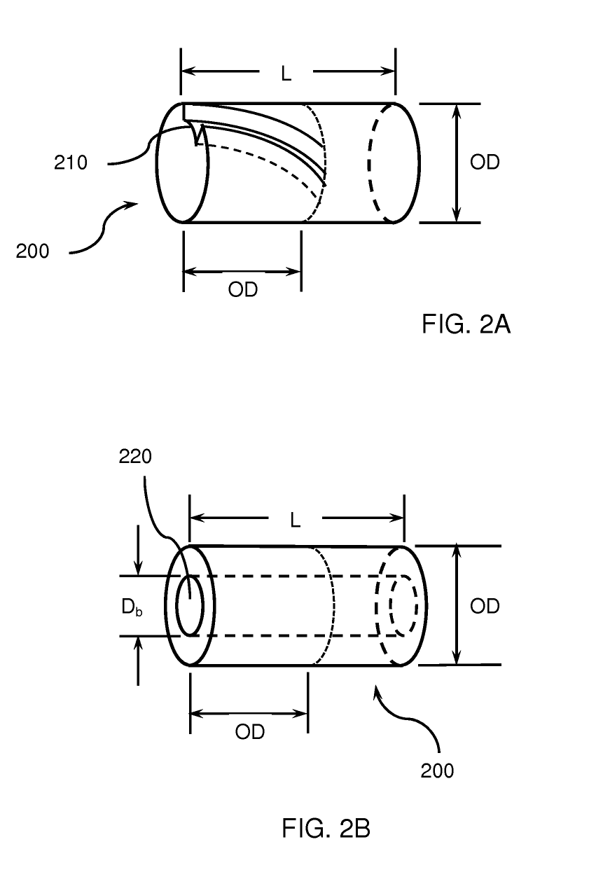

FIGS. 2A and 2B illustrate embodiments of packing material structures 200 comprising an external surface having an outside diameter, OD, and a length, L, where the packing material structure may be defined by an aspect ratio of the length to the OD. A unit body length may be equal to the outer diameter OD of the packing material structure when the structure is cylindrical, or the circumscribed OD if the structure is non-cylindrical.

In various embodiments, L/OD may be greater than 1 and less than 10, or alternatively L/OD may be greater than 1 and less than 5, or greater than 1 and less than 2.

In various embodiments, the OD of a packing material structure may be between about 1.0 mm and about 50 mm, or alternatively the OD may be between 1.0 mm and 25 mm, or between 1.0 mm and 10 mm, or between 2.0 mm and 10 mm, or between 5.0 mm and 8.0 mm.

As shown in FIG. 2A, one or more recess(es) 210 may be formed in the body of the packing material structure, so that the recess forms a channel below the surface of the packing material structure in various embodiments. In various embodiments, the recess forms a spiral around the outside of the packing material structure, where the recess may have an angle of rotation θₗ per unit body length. In various embodiments, the angle of rotation θₗ per OD may be between about 30° and 360°, or alternatively between 45° and 180°, or between 90° and 112.5°.

In embodiments of the present invention, at least one continuous recess may be formed in the external surface, and wherein the structure may be chemically active to absorb and/or catalyze chemical moieties that contact a surface of the support.

In various embodiments, the recess may rotate around the central axis of the body by an angle of rotation θ₂ per overall body length L, wherein θ₂ may be between about 45° and about 720°.

As shown in FIG. 2B, a hollow bore with a diameter, D_{b}, may be formed through the body of the packing material structure in various embodiments. The hollow bore may provide additional surface to the packing material structure for absorption and/or catalysis.

In various embodiments in which a packing material structure has a bore or a bore and a recess, there should be sufficient material forming a wall between the outside diameter of the hollow bore and the deepest section of the recess to maintain the structural integrity.

In various embodiments, T_{w} may be at least 20% of the OD of the packing material structure, or at least 33% of the OD of the packing material structure.

In some embodiments, the wall has a thickness, T_{w}, of not less than 1 mm, therefore when a packing material structure has an OD less than 2 mm, there may not be a hollow bore through the body because there may be insufficient wall thickness.

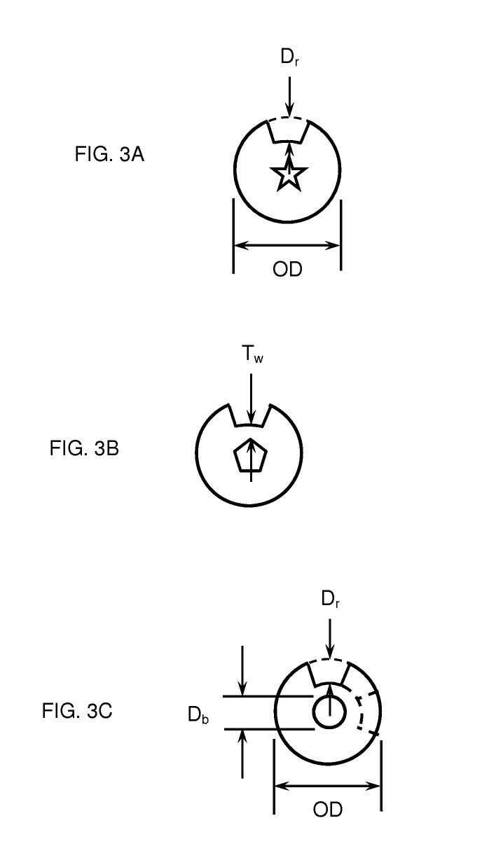

FIGS. 3A, 3B, and 3C illustrate embodiments of packing material structures having at least one recess (e.g., a channel) with a depth Dᵣ, and at least one hollow bore of various shapes through the packing material body, and a wall of thickness T_{w} between the inner-most edge of the recess and outer-most edge of the bore.

FIG. 3A depicts a cylindrical packing material structure having an OD and a single channel with a depth Dᵣ below the outer face of the cylinder, such that the distance from the center of the packing material structure to the recessed surface of the channel is less than the OD. The channel may have a helical angle around the packing material structure. The depicted cylindrical packing material structure also has a star-shaped bore positioned at the center of the structure.

In some embodiments the hollow bore may not be centered along the axis of the structure. In some embodiments the hollow bore may not go all the way through the structure.

FIG. 3B illustrates a cylindrical packing material structure with a single channel, and a pentagonal bore through the structure. The wall thickness T_{w} is measured between the recessed surface of the channel and the point of the bore closest to the recessed surface.

FIG. 3C illustrates a cylindrical packing material structure with a single channel that has a 90° angle of rotation over the length of the packing material structure, as shown in hidden lines, and a circular bore with a diameter D_{b} through the structure.

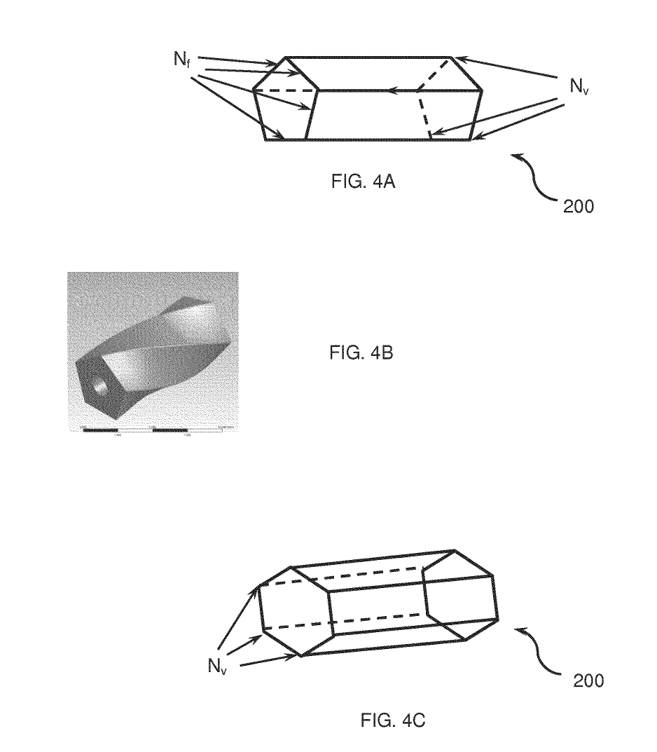

FIGS. 4A and 4C illustrate embodiments of packing material structures 200 with a geometric, transverse cross-sectional shape (e.g., triangular, square, rectangular, trapezoidal, pentagonal, hexagonal, polygonal) with N_{f} edges and Nᵥ vertices and an axis of extrusion of length L_{b}, wherein the vertices are a distance Rᵥ from the axis. The structures have a first face at a first end of the body and a second face at the second end of the body opposite the first end.

FIG. 4A illustrates a non-limiting example of a pentagonal packing structure with 5 edges and 5 vertices.

FIG. 4B illustrates a shaded view of the pentagonal packing material structure with a 45° helical twist.

FIG. 4C illustrates a non-limiting example of a hexagonal prism structure with 6 edges and 6 vertices.

In various embodiments the pentagonal prism structure may be twisted around the longitudinal axis, so that each longitudinal face experiences an angle of rotation θₗ per unit body length. In various embodiments, the angle of rotation θₗ per unit body length may be between about 30° and 360°, or alternatively between 45° and 180°, or between 90° and 112.5°. Twisting the longitudinal faces around the axis reduces the pressure drop per unit length of packed bed compared to a straight (i.e., prism) shape for the same number of sides.

In various embodiments, the body of the packing material structure may be formed by extruding a pliable material and cutting it to predetermined lengths.

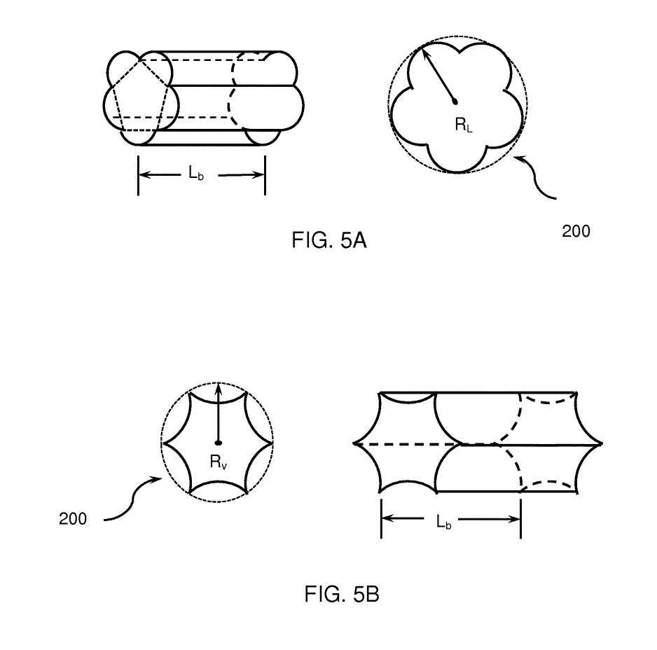

FIGS. 5A and 5B illustrate embodiments of packing material structures 200 with a transverse cross-sectional shape that has concave or convex edges, N_{f}, between a number, Nv, of vertices.

FIG. 5A illustrates an embodiment where the N_{f} edges are convex such that the body further comprises N₁ lobes between the Nᵥ vertices, whereas FIG. 5B illustrates an embodiment in which the N_{f} edges are concave such that the extruded body further comprises N_{c} channels or grooves between the Nᵥ vertices. The perimeter for each of the embodiments is greater than the circumference of the circle circumscribed around the outermost edge of the lobes of the convex embodiment with radius, R_{L}, or around the vertices of the concave embodiment with radius, Rᵥ.

In various embodiments, the lobes or groves may be twisted around the longitudinal axis, so that each lobe or grove experiences an angle of rotation θₗ per unit body length, where the unit body length is equal to the outer diameter OD of the circumscribed circle of the cross section. In various embodiments, the angle of rotation θₗ per OD may be between about 30° and 360°, or alternatively between 45° and 180°, or between 90° and 112.5°. Twisting the channels or lobes around the axis reduces the pressure drop per unit length of packed bed compared to a straight (i.e., prism) shape for the same number of sides and OD.

In embodiments, the angle of rotation may be a function of the number of vertices and edges, so that each edge and vertex rotates sufficiently to coincide with the next edge and vertex after advancing one OD in body length. For example, a packing material structure with five (5) edges and vertices may rotate by 180°/5=36° for each unit body length.

In an example of an embodiment, the angle of rotation θₗ per unit body length may be 90°, and the aspect ratio may be 4, so that the overall length of the body of the packing material structure is 4 times the OD and the lobes or groves make a full 360° rotation around the body.

In some embodiments, the packing material structure may be chemically active so it can absorb and/or catalyze chemical moieties that contact a surface of the packing material structure.

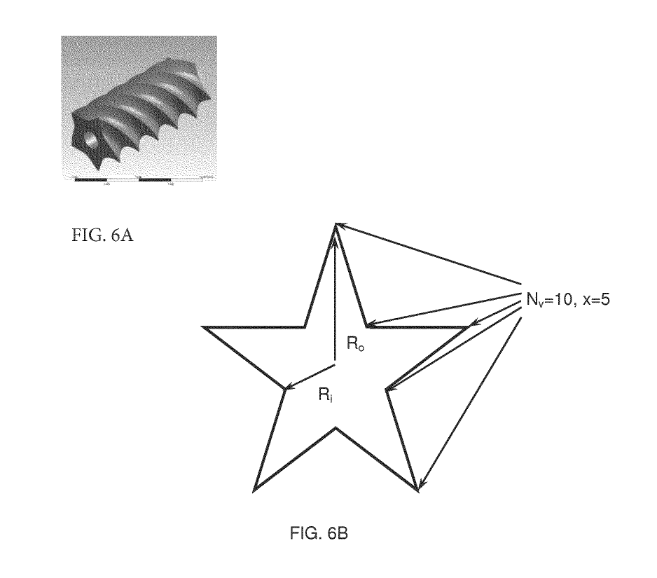

FIGS. 6A and 6B illustrate an embodiment of a packing material structure comprising a number, x, of projecting features, where x may be between 3 and 8. The packing material structure has Nᵥ=2x vertices, and N_{f}=2x edges, where the even-numbered vertices are located between the odd-numbered vertices and are a distance Rᵢ from the axis of the body and the odd-numbered vertices are a distance Rₒ from the axis of the body, where the edges between the vertices form N_{c}/2 channels with a channel depth D_{c}=Rₒ−Rᵢ. The vertices at Rᵢ are interior vertices around a minor radius that form the deepest point of the recess, whereas the vertices at Rₒ are exterior vertices around the major diameter or OD and form the farthest point of the recess. A shaded perspective view of an example of the twisted shape is also shown.

In various embodiments, the N_{f} edges connecting the Nᵥ vertices may be curved or straight.

In various embodiments, the channel depth D_{c} is from about 0.1 mm to about 3.0 mm.

The various embodiments of the packing material structures may have an aspect ratio (L/OD) of greater than 1 to about 10, or greater than 1 to about 5, or greater than 1 to about 4, or greater than 1 to about 2, or greater than 1 to about 1.5.

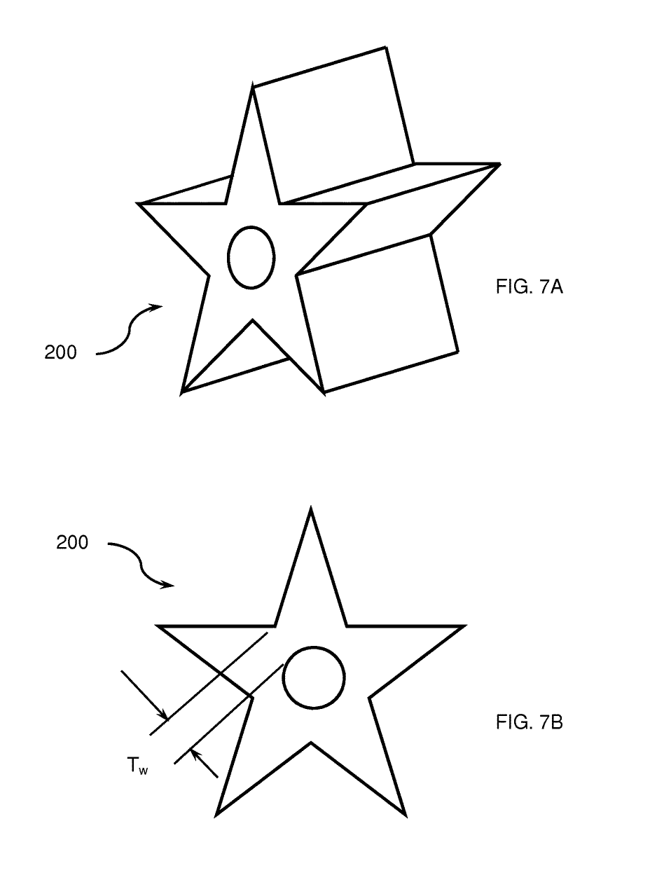

FIG. 7A illustrates an embodiment of a star-shaped prism with a circular bore through the center.

FIG. 7B illustrates an embodiment of a star-shaped prism with a wall thickness T_{w} between an interior vertex and the outside diameter of the circular bore. In various embodiments, the wall thickness T_{w} between the diameter of the hollow bore R_{b} and Rᵢ is at least 1 mm.

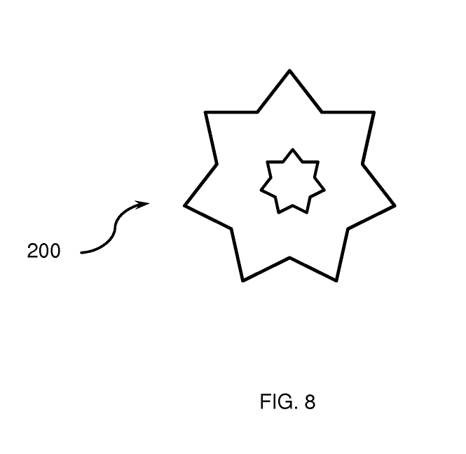

FIG. 8 illustrates another exemplary embodiment of a packing material structure with a 7-pointed star shaped cross-section, and a 7-pointed star shaped bore.

In various embodiments, the bore may have the same or a different shape than the cross-sectional shape of the packing material structure.

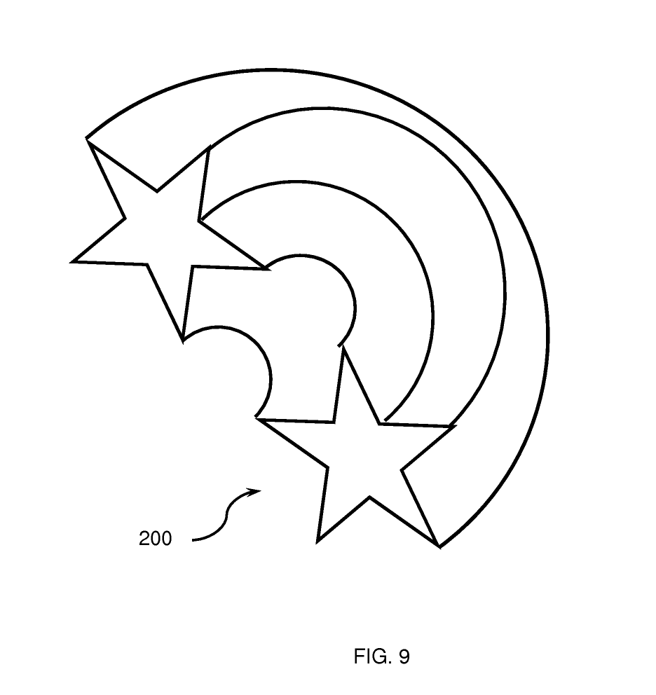

FIG. 9 illustrates an embodiment of a packing material structure with a 5-pointed star shaped cross-section and a body having a C-shaped curve.

In various embodiments, the packing material structure may have a 3-dimensional shape in which the body has a curved or helical axis.

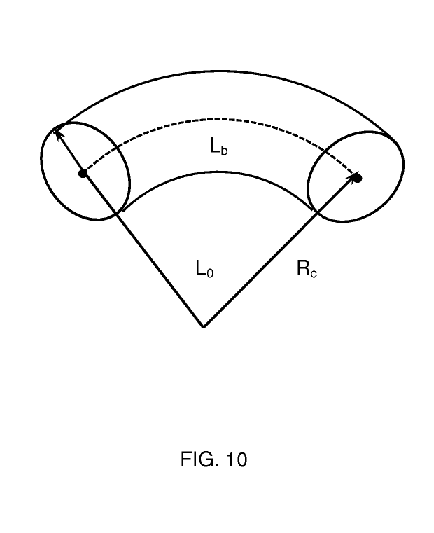

FIG. 10 illustrates an embodiment with a curved axis that may follow a predetermined radius, R_{c}, with an angle Lₒ between 30 and 180 degrees. The curved body of the packing material structure may have a length, L_{b}. The curve of the body may reduce or prevent the structures stacking or nesting.

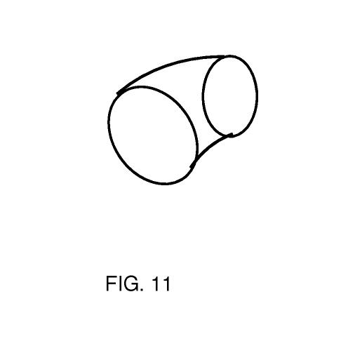

FIG. 11 illustrates a perspective of an embodiment with a curved axis and a varying diameter, where the angle L₀ is less than 45 degrees.

While the various embodiments of the present invention has been described as a packing material structure and/or catalyst support for fixed bed processes such as hydrotreating of petroleum distillation fractions and residues, it is to be understood that the packing material structures may be used more generally in other processes employing a packed bed of particles, as well as in processes employing ebullated catalyst beds, where the shapes, sizes, and configurations of the structures would reduce or prevent interlocking and clumping when fluidized.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A packed bed comprising:
a vessel comprising a shell, an inlet, and an outlet, wherein the space inside the shell between the inlet and outlet forms an internal volume;
a plurality of packing material structures filling at least a portion of the internal volume thereby forming a packed volume, wherein:
the packed volume has a void fraction,
the packing material structures provide an aggregate surface area, and
the packing material structures comprise a longitudinal axis, a cross-sectional shape, and a twisted shape around the longitudinal axis; and
the vessel has a pressure drop between the vessel inlet and vessel outlet, wherein the pressure drop is less than 1.0 times that of a packed bed of the packing material without the twisted shape.

2. The packed bed of claim 1, wherein the void fraction is less than about 0.8 and the pressure drop is less than 0.95 times that of a packed bed without the twisted shape.

3. The packed bed of claim 1, wherein the void fraction is less than about 0.8 and greater than about 0.45, and the pressure drop is less than 0.95 times that of a packed bed of the non-twisted shapes with the same cross-section and greater than about 0.3 times that of a packed bed of the packing material without the twisted shape.

4. The packed bed of claim 3, wherein the void fraction is less than about 0.8 and greater than about 0.55, and the pressure drop is less than 0.8 times that of a packed bed of the non-twisted shapes with the same cross-section and greater than about 0.3 times that of a packed bed of the packing material without the twisted shape.

5. The packed bed of claim 1, wherein the vessel is a tower, column, tank, drum, tube, pipe, or duct.

6-26. (canceled)

27. A geometrically shaped solid comprising:
a cylindrical body formed by a surface of revolution around a curved axis of revolution, wherein the surface of revolution is at a distance R₁ from the axis of revolution in a plane perpendicular from the axis of revolution;
one or more channels circumscribing a helical path around the cylindrical body, wherein the one or more channels have a crest at the surface of revolution of the cylindrical body and a trough within the cylindrical body, and the trough is at a distance R₂ from the axis of revolution; and
a length L_{b} extending from a first face of the cylindrical body to a second face of the cylindrical body, wherein the shaped solid is catalytically active.

28. The shaped solid of claim 27, wherein the axis of revolution has a varying radius R₀, that changes over an arc of angle L₀.

29. The shaped solid of claim 27, wherein the axis of revolution has a constant radius R₀, and circumscribes an arc of angle L₀.

30. The shaped solid of claim 27, wherein the cylindrical body has the shape of a segment of a torus with one or more channels formed around the body, such that the pitch between the channels along the outside edge of the torus is greater than the pitch along the inside edge of the torus.

31. The packed bed of claim 1, wherein the cross-sectional shape is a star shape.

32. The packed bed of claim 1, wherein the packing material structures comprise a catalytically active material comprising alumina.

33. The packed bed of claim 1, wherein the packing material structures comprise an extruded body.

34. A process for dehydrating alcohols, the process comprising:
exposing an alcohol to a packed bed reactor to form a dehydration product, the packed bed reactor comprising:
a vessel comprising a shell, an inlet, and an outlet, wherein the space inside the shell between the inlet and outlet forms an internal volume;
a plurality of packing material structures filling at least a portion of the internal volume thereby forming a packed volume;
wherein:
the packed volume has a void fraction;
the packing material structures provide an aggregate surface area;
the packing material structures comprise a longitudinal axis, a cross-sectional shape, and a twisted shape around the longitudinal axis; and
the vessel has a pressure drop between the vessel inlet and vessel outlet, wherein the pressure drop is less than 1.0 times that of a packed bed of the packing material without the twisted shape; and
collecting the dehydration product.

35. The process of claim 34, wherein the void fraction is less than about 0.8 and the pressure drop is less than 0.95 times that of a packed bed of the packing material without the twisted shape.

36. The process of claim 35, wherein the void fraction is less than about 0.8 and greater than about 0.45, and the pressure drop is less than 0.95 times that of a packed bed of the non-twisted shapes with the same cross-section and greater than about 0.3 times that of a packed bed of the packing material without the twisted shape.

37. The process of claim 36, wherein the void fraction is less than about 0.8 and greater than about 0.55, and the pressure drop is less than 0.8 times that of a packed bed of the non-twisted shapes with the same cross-section and greater than about 0.3 times that of a packed bed of the packing material without the twisted shape.

38. The process of claim 34, wherein the vessel is a tower, column, tank, drum, tube, pipe, or duct.

39. The process of claim 38, wherein the alcohol comprises an aliphatic alcohol or an aryl alcohol.

40. The process of claim 34, wherein the cross-sectional shape is a star-shape.

41. The process of claim 34, wherein the dehydration product is an olefin.

42. A process for modification of an organic material, the process comprising:
exposing an organic material to a packed bed reactor to form a modified organic material product, the packed bed reactor comprising:
a vessel comprising a shell, an inlet, and an outlet, wherein the space inside the shell between the inlet and outlet forms an internal volume;
a plurality of packing material structures filling at least a portion of the internal volume thereby forming a packed volume
wherein:
the packed volume has a void fraction;
the packing material structures provide an aggregate surface area;
the packing material structures comprise a longitudinal axis, a cross-sectional diameter, and a twisted shape around the longitudinal axis; and
the vessel has a pressure drop between the vessel inlet and vessel outlet, wherein the pressure drop is less than 1.0 times that of a packed bed of the packing material without the twisted shape; and
collecting the modified organic material product;
wherein: the exposing comprises cracking, reforming, hydrogenating, oxidizing, dehydrogenating, dehydrating, polymerizing, alkylating or dealkylating aryl compounds, isomerizing, or hydrodesulfurizing the organic material into the modified organic material product.

43. The process of claim 42, wherein the cross-sectional shape is a star-shape.
